(19) United States
(12) Patent Application Publication
(10) Pub. No.: US 2009/0112037 A1
(43) Pub. Date: Apr. 30, 2009

(54) Process for Recovering Ethylene From an Autothermal Cracking Reactor Effluent

(76) Inventors: Rian Reyneke, Katy, TX (US); Michael J. Foral, Aurora, IL (US); Graeme Parker, Falkirk (GB); Wayne W.Y. Eng, League City, TX (US)

Correspondence Address: NIXON & VANDERHYE, PC, 901 NORTH GLEBE ROAD, 11TH FLOOR, ARLINGTON, VA, 22203, US

(21) Appl. No.: 11/922,761

(22) PCT Filed: Jul. 28, 2005
(86) PCT No.: PCT/US2005/026763

§ 371 Date: Dec. 21, 2007

(51) Int. Cl. C07C 4/04

(52) U.S. Cl. 585/648

(57) ABSTRACT

The process of this invention represents an improved method for recovering a purified ethylene product and optionally a purified hydrogen product from the effluent of an autothermal cracking reactor. The process consists of cracked gas chilling, rough separation of a hydrogen-rich stream, demethanization, separation of ethylene from the demethanizer bottoms product, and final purification of the ethylene product. Hydrocarbons heavier than ethylene, including ethane, propylene, and propane are recycled to the ATC reactor. Optionally a purified hydrogen product can be obtained from the hydrogen-rich stream. The invention is particularly useful when the fresh hydrocarbons feed to the autothermal cracking reactor is ethane or a mixture of ethane and propane.

### STATEMENT REGARDING FEDERALLY SPONSORED RESEARCH OR DEVELOPMENT

This invention was made with government support under United States Department of Energy Cooperative Agreement No. DE-FC07-01ID 14090.

### PROCESS

1. Field Of the Invention

This invention relates to the recovery of ethylene and optionally hydrogen from the effluent of an autothermal cracking reactor.

2. Background

The production of ethylene (and other olefins) from a paraffinic hydrocarbon feed by steam cracking is well-known in the art. In a typical steam cracking process a paraffinic hydrocarbon feed such as ethane, propane, butane, naphtha, gas oil, or a combination of these feeds is introduced, along with a controlled amount of steam, into a cracking furnace, in which the steam/paraffinic hydrocarbon mixture is heated to high temperatures and the paraffinic hydrocarbon feed is thermally cracked to yield a range of products.

This cracking reaction typically takes place under relatively low pressure, usually below 25 psig.

The cracked gas composition depends on the paraffinic hydrocarbon feed composition, but generally comprises hydrogen, methane, ethylene, ethane, propylene, propane, higher hydrocarbons, and water vapor. Small amounts of other products, such as acetylene, propadiene, methyl acetylene, carbon monoxide and carbon dioxide are also present.

The ethylene and any other desired products must thus be separated from the cracked gas.

Typically, the hot cracked gas mixture from the cracking furnace is first cooled, for example by directing it to a transfer line exchanger in which it is cooled by raising high pressure steam. Further cooling can be achieved by various means, including boiler feed water preheating, oil quenching, and water quenching.

The quenched cracked gas may then be compressed to relatively high pressure, typically around 200-500 psig, before carbon dioxide and any remaining water is removed and the compressed, dried cracked gas mixture is directed to the separation section of the plant.

There are many methods by which the compounds in the cracked gas can be separated to yield olefin product(s), fuel gas, hydrogen, recycle streams, and heavy products. Some suitable methods are described in Hydrocarbon Processing, March 2003, pp. 96-99.

In general, however, the separation methods typically fall into one of three types.

First are the front-end demethanizer methods, in which the first step of separation is to chill the cracked gas and remove the methane and lighter components from the remaining heavier hydrocarbons.

Front-end deethanizer methods chill the cracked gas and then remove the ethane and lighter components from the remaining heavier components.

Front-end depropanizer flowsheets are similar, except that the first step is to remove the propane and lighter components from the heavier hydrocarbons.

The optimal separation method will depend on many factors, including feed type, product requirements, energy cost, and feed cost, among others.

An alternative method of production of olefins from a paraffinic hydrocarbon feed is autothermal cracking, as described for example, in EP 332289B; EP-529793B; EP-A-0709446 and WO 00/14035. In an autothermal cracking process a paraffinic hydrocarbon-containing feedstock is mixed with oxygen and passed over an autothermal cracking catalyst. The autothermal cracking catalyst is capable of supporting combustion beyond the fuel rich limit of flammability. Combustion of a portion of the feed is initiated on the catalyst surface and the heat required to raise the reactants to the process temperature and to carry out the endothennic cracking process is generated in situ.

More recently, it has been found that autothermal cracking processes can tolerate significant levels of unsaturated compounds in the feed to the process, as described in WO 2004/087626.

In contrast, for steam cracking it is well-known that unsaturated hydrocarbons have a high propensity to cause carbonaceous fouling of the furnace process equipment. Thus, it is undesirable to recycle high levels of olefinic materials to a conventional steam cracker (low levels of unsaturated hydrocarbons are inherently present in many hydrocarbon feeds, and it is undesirable to add to these).

Thus, whilst conventional ethylene recovery and purification (separation) methods have included steps to ensure that olefinic materials are largely removed from any stream that is to be recycled to the steam cracker, we have found that the ethylene product separation steps for the cracked product from an autothermal cracking process may be operated with significantly reduced energy requirement by allowing olefinic materials to be present in the hydrocarbon recycle streams to an autothermal cracking process. This energy reduction allows the autothermal cracking-based manufacture of ethylene to be achieved in a more economic manner, and in particular provides a reduced refrigeration duty for the separations. Overall yields of ethylene may also be improved by the recycle of unsaturated hydrocarbons other than ethylene, since these may be expected to preferentially combust (in preference to the paraffinic hydrocarbon feed) in the autothermal cracking reaction.

Thus, in a first aspect, the present invention provides a process for the production of ethylene from a paraffinic hydrocarbon-containing feed, said process comprising the steps of:

(i) autothermally cracking the paraffinic hydrocarbon-containing feed with a molecular oxygen-containing gas in contact with a catalyst capable of supporting combustion beyond the normal fuel rich limit of flammability to produce a product stream comprising ethylene, ethane, propylene, carbon monoxide, hydrogen, and hydrocarbons heavier than propylene,
(ii) chilling and partially condensing said product stream to produce one or more liquid streams, and an uncondensed vapor stream;
(iii) treating at least one of said one or more liquid streams in a demethanizer distillation column, to produce an overhead product comprising hydrogen, carbon monoxide and methane and a bottoms product comprising hydrocarbons heavier than methane;
(iv) treating the bottoms product comprising hydrocarbons heavier than methane from step (iii) in a deethyleneizer column, to produce an overhead product comprising ethylene, ethane, propylene and propane, with essentially no C4 hydrocarbons, and a bottoms product comprising C4 and heavier hydrocarbons,
(v) treating the overhead product comprising ethylene, ethane, propylene and propane in a C2 splitter to produce an overhead ethylene product stream and a bottoms stream comprising ethane, propylene and propane, and
(vi) recycling the bottoms stream comprising ethane, propylene and propane to the autothermal cracking step (i).

In step (i) of the process of the present invention, a paraffinic hydrocarbon-containing feed is autothermally cracked with a molecular oxygen-containing gas in contact with a catalyst capable of supporting combustion beyond the normal fuel rich limit of flammability to produce a product stream comprising ethylene, ethane, propylene, carbon monoxide, hydrogen, and hydrocarbons heavier than propylene. Other products, such as water vapor and carbon dioxide may also be present, as may be unreacted oxygen.

The catalyst capable of supporting combustion beyond the fuel rich limit of flammability usually comprises a Group VIII metal as its catalytic component. Suitable Group VIII metals include platinum, palladium, ruthenium, rhodium, osmium and iridium. Rhodium, and more particularly, platinum and palladium are preferred. Typical Group VIII metal loadings range from 0.01 to 100 wt %, preferably, between 0.01 to 20 wt %, and more preferably, from 0.01 to 10 wt % based on the total dry weight of the catalyst.

Where a Group VIII catalyst is employed, it is preferably employed in combination with a catalyst promoter. The promoter may be a Group IIIA, IVA, and/or VA metal. Alternatively, the promoter may be a transition metal; the transition metal promoter being a different metal to that which may be employed as the Group VIII transition metal catalytic component.

The paraffinic hydrocarbon-containing feed may comprise liquid or gaseous paraffinic hydrocarbons. Suitable liquid feeds include naphtha, gas oils, vacuum gas oils and mixtures thereof. Preferably, however, gaseous feeds such as ethane, propane, butane and mixtures thereof are employed.

The paraffinic hydrocarbon-containing feedstock may be fed with any suitable molecular oxygen-containing gas. Suitably, the molecular oxygen-containing gas is molecular oxygen itself, air and/or mixtures thereof. The molecular oxygen-containing gas may be mixed with an inert gas such as nitrogen or argon.

Preferably the paraffinic hydrocarbon-containing feedstock and molecular oxygen-containing gas are fed to the autothermal cracker at a ratio of paraffinic hydrocarbon to molecular oxygen of 5 to 16 times, preferably 5 to 13.5 times, more preferably 6 to 10 times, the stoichiometric ratio of paraffinic hydrocarbon to molecular oxygen required for complete combustion of the hydrocarbon to carbon dioxide and water.

The paraffinic hydrocarbon is passed over the catalyst at a gas hourly space velocity of greater than 10,000 h⁻¹, preferably above 20,000 h⁻¹ and most preferably, greater than 100,000 h⁻¹. It will be understood, however, that the optimum gas hourly space velocity will depend upon the pressure and nature of the feed composition.

Additional feed components may be co-fed into the autothermal cracker, such as hydrogen, carbon monoxide, carbon dioxide or steam.

The autothermal cracking process is suitably carried out at a catalyst exit temperature in the range 600° C. to 1200° C., preferably, in the range 850° C. to 1050° C. and, most preferably, in the range 900° C. to 1000° C. To avoid further reactions taking place, the ATC product stream should be rapidly cooled, typically by cooling to between 750-600° C. within 20 milliseconds of formation. Advantageously wherein the autothermal cracking process is operated at a pressure of greater than 20 barg the products are cooled to between 750-600° C. within 10 milliseconds of formation.

To produce the product stream comprising ethylene, ethane, propylene, carbon monoxide, hydrogen, and hydrocarbons heavier than propylene for subsequent separation, the cracked gas stream from the autothermal cracker is first treated to remove carbon dioxide, for example, using an amine-based absorption system such as MEA or TEA (or mixtures of both), or other commercially available CO₂ removal process.

Any residual oxygen may be removed next, typically by contact with a suitable catalyst.

Any residual water may be removed next. Any suitable drying process may be used, for example by use of a suitable molecular sieve.

All or a portion of this ATC product stream (after carbon dioxide removal and after oxygen and/or water removal) may then be passed as the product stream comprising ethylene, ethane, propylene, carbon monoxide, hydrogen, and hydrocarbons heavier than propylene to the subsequent separation steps of the first aspect of the present invention.

In step (ii) of the process of the present invention, the product stream comprising ethylene, ethane, propylene, carbon monoxide, hydrogen, and hydrocarbons heavier than propylene is subjected to chilling and partial condensation to produce one or more liquid streams, and an uncondensed vapor stream;

This may be achieved by passing the product stream comprising ethylene, ethane, propylene, carbon monoxide, hydrogen, and hydrocarbons heavier than propylene to a chilling train comprising one or more stages in which chilling and partial condensation occur.

Within each stage of the chilling train, the chilling and partial condensation are followed by liquid and vapor separation. The vapor is passed to the next chilling/partial condensation stage (if any), the uncondensed vapor stream (from the final chilling/partial condensation stage) forming a stream comprising carbon monoxide, hydrogen, and methane. The uncondensed vapor stream comprises hydrogen, carbon monoxide and methane and may be used as a fuel gas, may be further treated to recover hydrogen and/or carbon monoxide and/or may be used as a synthesis gas feed for a subsequent chemical process.

The liquid stream removed from each stage forms the one or more liquid streams. The one or more liquid streams generally comprises methane, ethylene, ethane and heavier hydrocarbons.

In step (iii) of the process of the present invention at least one of said one or more liquid streams is treated in a demethanizer distillation column, to produce an overhead product comprising hydrogen, carbon monoxide and methane and a bottoms product comprising hydrocarbons heavier than methane.

Where there are more than one liquid streams produced in step (ii) preferably all of said streams are passed to the demethanizer column in step (iii).

Where there are more than one liquid streams produced in step (ii) that are passed to the demethanizer column in step (iii) they may be passed to the demethanizer as separate streams, or two or more of the liquid streams may be combined and passed to the demethanizer distillation column.

The overhead product comprising hydrogen, carbon monoxide and methane may be combined with the uncondensed vapor stream recovered from the chilling and partial condensation in step (ii) or may separately be used, for example as a fuel gas, treated to recover hydrogen and/or carbon monoxide and/or used as a synthesis gas feed for a subsequent chemical process.

In step (iv) the bottoms product comprising hydrocarbons heavier than methane is treated in a deethyleneizer column, to produce an overhead product comprising ethylene, ethane, propylene and propane, with essentially no C4 hydrocarbons, and a bottoms product comprising C4 and heavier hydrocarbons. In a conventional separations train for a steam cracking process, this column is a deethanizer column, and the overhead stream would comprise essentially only ethylene and ethane, any propane and propylene being removed from the column as components of the bottoms product. The separation according to the process of the present invention is significantly less energy intensive than conventional separation in a deethanizer column. This may be achieved by taking advantage of the fact that a less energy intensive separation may be utilized and the propane and propylene in the overhead may subsequently be separated in an otherwise conventional C2 splitter, and recycled to the autothermal cracking process with ethane.

The overhead product form the deethyleneizer may contain a small amount of acetylene. If so, this stream may be directed to an acetylene hydrogenation system prior to treatment in the C2 splitter of step (v).

Preferably, at least a portion of the bottoms product of the deethyleneizer column may also be recycled to the autothermal cracking process of step (i). Typically, the bottoms stream is treated to remove any undesirable heavy components, and the remaining components recycled.

In step (v), the deethyleneizer overhead product comprising ethylene, ethane, propylene and propane is treated in a C2 splitter to produce an overhead ethylene product stream and a bottoms stream comprising ethane, propylene and propane. In step (vi) of the process of the present invention, this bottoms stream is recycled to the autothermal cracking process of step (i).

Using conventional separation steps for steam cracking, the C2 splitter bottoms stream would comprise essentially only ethane. The process of the present invention takes advantage of the fact that an autothermal cracking process can tolerate propylene in a recycle without increased tendency for coking.

In addition, the recycled propylene may be expected to combust in the autothermal cracking process in preference to the recycled ethane and propane, and/or in preference to the paraffinic hydrocarbon of the paraffinic hydrocarbon-containing feed, reducing the amount of paraffinic hydrocarbon which needs to be combusted to generate the heat for cracking and hence, improved overall yields of ethylene may be obtained by the process of the present invention.

We have found that the process of this invention is particularly beneficial when the feed to the ATC reactor is relatively light, containing primarily ethane, propane, or butanes. In this case relatively little propylene is produced in the ATC reactor and a purified propylene product typically cannot be economically recovered. When the feed to the ATC reactor is heavier, for example a naphtha or heavier hydrocarbon, it may become economical to recover a purified propylene product.

### DESCRIPTION OF FIGURES

The invention will now be illustrated with respect to the following Figures and Example, wherein:

FIG. 1 depicts a preferred embodiment of this invention,

FIG. 2 depicts an alternate arrangement for chilling the cracked gas and recovering the ethylene therefrom through the use of rectification, and

FIG. 3 depicts an alternate arrangement for the deethyleneizer column which requires less energy.

It should be noted that details of some conventional process equipment, such as vapor-liquid separation drums, process control valves, pumps and the like have been omitted for clarity.

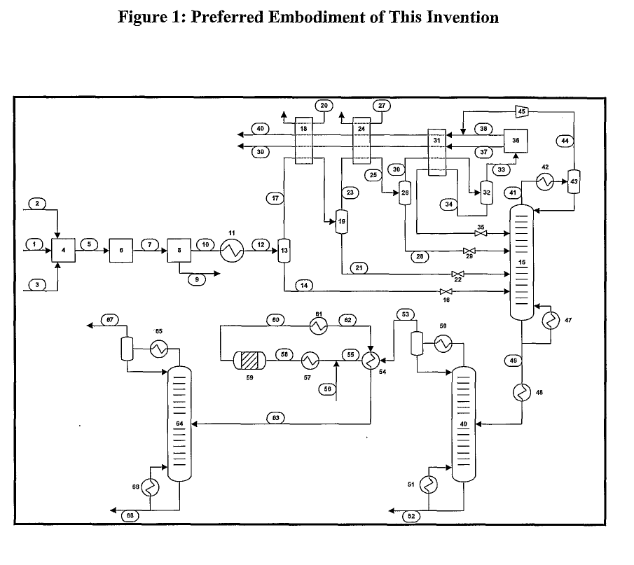

FIG. 1 depicts one embodiment of this invention. It should be noted that details of some of the elements of FIG. 1, in particular the autothermal reactor, thermal quench, and gas conditioning sections, have been omitted in order to more clearly illustrate the concepts of the present invention.

In the process of FIG. 1, a hydrocarbon feed, stream 1, a hydrogen co-feed, stream 2 and a molecular oxygen-containing gas, stream 3, enter the autothermal cracking reactor 4. Stream 1 could in practice represent a number of distinct streams of different compositions, including, for example, fresh feed streams and recycle streams. Within the autothermal cracking reactor the components are reacted to form a hot effluent gas 5 which typically comprises ethylene, ethane, propylene, methane, carbon monoxide, carbon dioxide, hydrogen, water vapor and hydrocarbons heavier than propylene. This gas is rapidly quenched by generating high-pressure superheated steam in the quench section 6. Within the quench section the majority of the water vapor is also removed, typically through use of a water quench tower.

The cooled reactor effluent stream 7 then enters the gas conditioning section 8. In this section impurities such as carbon dioxide, residual molecular oxygen, and traces of water vapor are removed. This section typically contains an amine or caustic tower or other means for removing carbon dioxide, catalytic beds for removal of residual amounts of oxygen, and process dryers for removal of water vapor. If the autothermal reactor is operated at a relatively low pressure, for example below about 10 barg, this section would also typically contain cracked gas compressors. A condensed heavy hydrocarbon stream may be generated in either section 6 or section 8, indicated by stream 9. Such a stream could be further processed as desired, recycled to the ATC reactor, or used as fuel.

The cooled, conditioned cracked gas stream 10 is chilled and partially condensed in exchanger 11. The liquid and vapor in the resulting stream 12 are separated in drum 13. The liquid stream 14 is directed to the demethanizer column 15. If the pressure of the demethanizer column is substantially lower than that of drum 13 a means for reducing the pressure of stream 14 can be provided, such as valve 16.

The vapor stream 17 is chilled and partially condensed in exchanger 18, and the resulting vapor and liquid separated in drum 19. The refrigeration for the chilling in exchanger 18 is provided by reheating of process gases as described below, and optionally by an external refrigerant stream, shown as stream 20. The liquid stream 21 is directed through valve 22 to demethanizer column 15.

The vapor stream 23 is further chilled and partially condensed in exchanger 24, and the liquid and vapor in the resulting mixed phase stream 25 are separated in drum 26. The refrigeration for the chilling in exchanger 24 is provided by reheating of process gases as described below, and optionally by an external refrigerant stream, shown as stream 27. The liquid 28 is directed through valve 29 to demethanizer column 15.

The external refrigeration streams 20 and 27 could be generated by any of a number of external refrigeration circuits, including pure component circuits, mixed refrigerant circuits, gas expansion circuits, and the like. We have found that the process of this invention exhibits particular benefits with respect to reduced construction requirements and enhanced energy savings when the refrigeration requirements are provided by a combination of a propylene refrigeration system for the warmer refrigeration levels, and a mixed refrigeration system for the colder levels. For the embodiment of FIG. 1, for example, propylene refrigerant could be utilized in exchangers 11, 48, 50, and 65 and possibly others. The mixed refrigeration system would then provide at least a portion of the refrigeration for exchangers 18, 24, and 42 and possibly others. Those skilled in the art will recognize that there are many viable designs for a mixed refrigeration system that would provide the required duties. Likewise, there are many different mixed refrigerant compositions that could be used as the working fluid. Typically the mixed refrigerant would contain, but not be limited to, C1 to C3 hydrocarbons and additionally other light and heavy components to tailor the boiling behavior of the refrigerant mixture. All such details of the mixed refrigeration system design are within the scope of this invention.

In a mixed refrigerant system the working fluid is composed of more than one component. For example, the working fluid can be composed of a mixture of hydrocarbons such as methane, ethane, ethylene, propane, propylene, butane, or butene. In a closed-loop mixed refrigerant system, the working fluid is compressed, cooled and at least partially condensed, and then flashed to lower pressure whereupon any remaining liquid is vaporized to provide refrigeration at temperatures below ambient temperature. The relatively low-pressure vaporized mixed refrigerant working fluid is then recycled to the compressor to be re-compressed.

The vapor stream 30 is chilled and partially condensed in exchanger 31, and the liquid and vapor in the resulting mixed phase stream are separated in drum 32. Typically the temperature of drum 32 will be controlled so that there is a relatively small amount of ethylene exiting in the vapor stream 33. The liquid stream 34 is reheated in exchanger 31 and then directed through valve 35 to the demethanizer column 15. Exchanger 31 advantageously operates without the need for refrigerant generated by an external refrigeration cycle.

FIG. 1 therefore describes a process in which the cooled, conditioned cracked gas stream 10 undergoes a total of four separate chilling and partial condensation steps. It should be noted that the number of chilling and partial condensation steps may be more or fewer than four. The optimal number of steps depends on a number of factors, including feed type, reactor effluent composition, and the commercial and economic goals of the process.

The vapor stream 33 can be directed to a hydrogen recovery unit 36. Optionally, a portion of this stream could be expanded across a valve or turboexpander and the resulting cold stream reheated in the hydrogen recovery unit and exchangers 31, 24, and 18 to provide refrigeration for these steps. Typically a cryogenic adiabatic hydrogen recovery unit will be used in step 36. The design of these units is well known by those skilled in the art and is not presented in detail here.

Such adiabatic hydrogen recovery units produce at least two streams, one a relatively high-pressure hydrogen product stream 37, and the other a relatively low-pressure stream 38 which comprises methane, carbon monoxide, and hydrogen. These streams exit the hydrogen recovery unit 36 at a relatively cold temperature. They are reheated in exchangers 31, 24, 18, and optionally 11. The warmed high-pressure hydrogen product stream 39 can be further warmed elsewhere in the process and can be either sold as hydrogen product, recycled to the reactor as stream 2, or provided for other suitable uses. The warmed low-pressure stream, stream 40, can also be further warmed elsewhere in the process and is typically used as fuel.

The gross overhead vapor from the demethanizer column 15, stream 41, is partially condensed in exchanger 42. Liquid and vapor in the resulting stream are separated in reflux drum 43. The liquid is returned to the top of the demethanizer as reflux liquid. The net overhead vapor of the demethanizer column, stream 44, comprises methane, carbon monoxide and some residual hydrogen. It can be directed through an expander 45 and then joined with the low-pressure stream 38 if desired and reheated in exchangers 34, 21, and 18 as shown. Typically the demethanizer column will be operated so that there is little ethylene in the net overhead vapor stream 44.

The bottoms product of the demethanizer, stream 46, comprises hydrocarbons heavier than methane, primarily ethylene, ethane, and hydrocarbons heavier than ethane. In the demethanizer the lighter components are stripped from the bottoms liquid with stripping vapor generated by reboiler 47. Advantageously the heat for this reboiler is supplied through the chilling of a refrigeration or process stream. For example, the reboiler 47 could be integrated into exchanger 31 in order to provide additional chilling in this exchanger.

Stream 46 can be heated or cooled in exchanger 48 and directed to an intermediate location on the deethyleneizer column 49. The deethyleneizer column is refluxed using partial condenser 50 and reboiled with exchanger 51. The bottoms stream 52 comprises predominantly C4 and heavier hydrocarbons, but may also contain some propane and propylene, ethane, and only a small amount of ethylene, for example, less than about 0.1 mol % ethylene. This steam is recycled to the autothermal cracking reactor 4. It may be desirable to remove the heaviest components from this stream before it is recycled to the autothermal cracking reactor.

The net overhead product of the deethyleneizer column, stream 53, comprises primarily ethylene, as well as ethane, propylene, and propane. The column is operated in such a way that there are essentially no C4 hydrocarbons, for example less than about 300 ppm of 1,3 butadiene, in stream 53.

Stream 53 may contain a small amount of acetylene. If so, this stream may be directed to an acetylene hydrogenation system. A typical hydrogenation system is shown in FIG. 1. Those skilled in the art will understand that many options exist for the design of the acetylene hydrogenation system. All such specific acetylene hydrogenation system designs are included within the scope of this invention. Stream 53 is first warmed by heat exchange with hydrogenation reactor effluent gases in exchanger 54. The warmed stream 55 is then mixed with a controlled amount of purified hydrogen as stream 56 and the combined stream is heated to reaction temperature, in exchanger 57. The purified hydrogen stream 56 may be obtained from the hydrogen product stream 39 if desired. In this case additional purification such as with a pressure-swing adsorption (PSA) system may be required to remove impurities from the hydrogen stream. The heated stream 58 is sent to acetylene reactor 59 where the majority of the acetylene is converted to ethylene and/or ethane via catalytic hydrogenation. In practice there may be more than one reactor in series with intercooling, depending on the level of acetylene in stream 53 and the desired acetylene conversion. These factors are well known to those skilled in the art.

The reactor effluent stream 60 is cooled, typically against cooling water, in exchanger 61 and the resulting cooled stream 62 is further cooled in exchanger 54. The final cooled stream 63 is fed to an intermediate location on C2 splitter column 64. The C2 splitter is refluxed with condenser 65 and reboiled with exchanger 66. The net overhead product, stream 67, comprises the final ethylene product. Stream 67 can be in either vapor or liquid form. The bottoms product, stream 68, comprises ethane and any hydrocarbons heavier than ethane that enter the column (i.e. propylene and propane). This stream is recycled to the autothermal cracking reactor 4.

In a conventional ethylene purification system for processing the effluent of a steam cracking furnace, the deethanizer overhead stream, corresponding to stream 53 of FIG. 1, would have a relatively low concentration hydrocarbons heavier than ethane such as propylene. In such a case the bottoms of the C2 splitter, stream 68 of FIG. 1, would therefore be relatively pure ethane which could then be recycled to the steam cracker furnaces. For example, the conventional ethylene plant configuration described by Manley and Hahesy (Hydrocarbon Processing, April 1999, pp. 117-124) is typical of existing processes wherein the deethanizer is operated so that the molar ratio of C3 components (particularly propylene) to ethane in the deethanizer overhead is less than 0.005. This stream, when processed in a downstream C2 splitter column, produces a C2 splitter bottoms stream containing 0.5 mol % propylene, which is typically recycled to the cracking furnaces.

The ratio of propylene to ethane in a conventional deethanizer tower is controlled at a relatively low level because the presence of significant amounts of propylene in the C2 splitter bottoms would be detrimental to the operation of conventional cracking furnaces, causing a high propensity to coke formation in the furnaces. An autothermal cracking reactor, on the other hand, is not adversely affected by the presence of propylene in this stream. Therefore, in contradiction to conventional practice, the molar ratio of propylene to ethane in the deethyleneizer tower overhead of this invention, and therefore in the C2 splitter feed, is well above 0.005. This novel combination of a relatively propylene-rich deethyleneizer column overhead stream coupled with an autothermal cracking reactor provides a means for recovering the desired ethylene product with significantly lower energy use and reduced construction requirements, while avoiding detrimental effects in the ethylene production reactor.

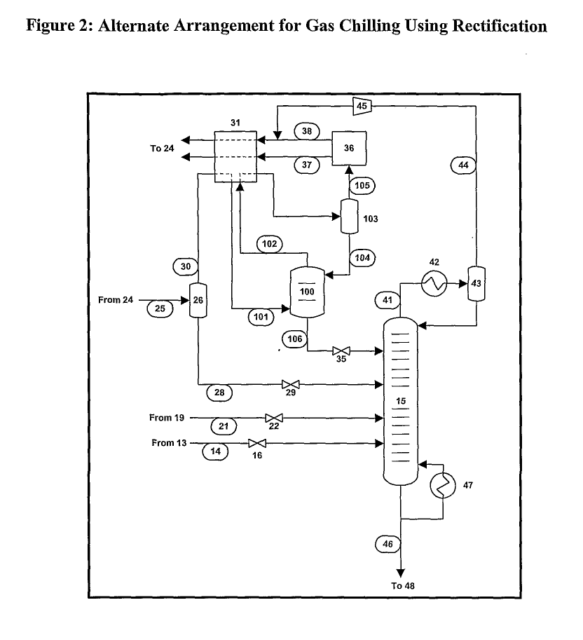

FIG. 2 depicts an alternate method for chilling the cracked gas and recovering ethylene from the uncondensed vapor. In this figure many of the streams and most of the equipment are the same as in FIG. 1 and therefore have the same number identifier. In the arrangement of FIG. 2, rather than employing only chilling, partial condensation, and vapor/liquid separation to recover ethylene from the uncondensed gas, a rectification operation is included in the cracked gas chilling train. In such a rectification operation vapor which is relatively rich in heavier components is contacted in a countercurrent manner with downflowing liquid. In this way the heavier components of the vapor are at least partially rectified into the downflowing liquid. At the same time the downflowing liquid is at least partially stripped of lighter material. The use of rectification in the chilling train provides for a more efficient separation of ethylene from the remaining uncondensed vapor, and typically requires less energy to achieve the desired level of separation.

The arrangement depicted in FIG. 2 employs a single rectification zone 100. The vapor stream 30 from drum 26 is chilled in exchanger 31 to produce the partially condensed stream 101. This stream enters the bottom of rectifier 100. This rectifier contains a small number of equilibrium stages, typically less than 10 theoretical stages. The overhead vapor 102 is further chilled and partially condensed in a colder section of exchanger 31 and the resulting liquid and vapor separated in drum 103. The liquid stream 104 is directed to the top of rectifier 100 as reflux liquid. The vapor stream 105 typically comprises hydrogen, methane and CO and is sent to the hydrogen recovery unit 36, similar to stream 33 of FIG. 1. The bottoms stream 106 is directed to demethanizer 15 through valve 35. In the method of FIG. 2 it is particularly advantageous to combine the demethanizer reboiler 47 with exchanger 31 so that some of the chilling duty within exchanger 31 is provided through the reboiling of demethanizer bottoms material.

Many design options exist for utilizing rectification within the chilling train shown in FIG. 1. In FIG. 2 a single rectification zone is utilized. Alternatively, this separation could also operate by combining rectification and chilling operations. Such combined chilling and rectification options would include, but not be limited to, a dephlegmator, a distillation column with side condensers, or the advanced rectification designs of U.S. Pat. No. 6,343,487 and U.S. Pat. No. 4,496,381, among others.

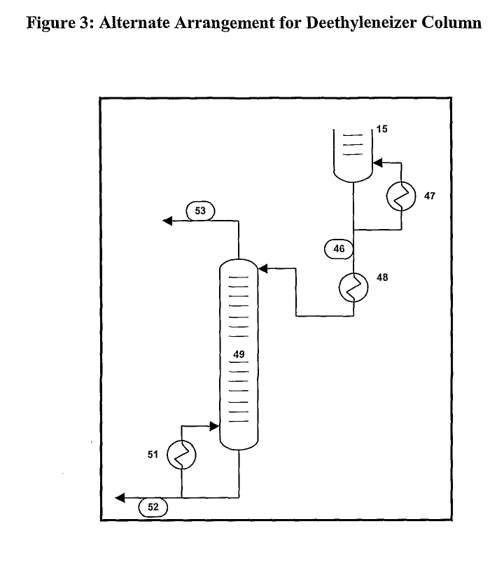

FIG. 3 depicts an alternate arrangement of the deethyleneizer column 49. In this arrangement the deethyleneizer operates as a stripper column, with no rectification section above the feed and therefore no condenser. This design uses even less refrigeration power than the embodiment of FIG. 1, and because there are fewer pieces of equipment the installed cost will be somewhat lower. In the embodiment of FIG. 3, stream 53 comprises ethylene, ethane and C3 components and stream 52 comprises C3 and heavier hydrocarbons. If any C4 or heavier hydrocarbons are present in stream 46 a portion of them will also enter the overhead vapor stream 53. In this case the catalyst in the acetylene reactor 59 (shown in FIG. 1) must be able to tolerate such C4 and heavier hydrocarbons in the feed. If such a catalyst is used, or if there are few C4 hydrocarbons in the deethyleneizer feed, then the arrangement of FIG. 3 may be preferred.

### EXAMPLE

An ethylene recovery and purification system based on the embodiment of FIG. 1 was simulated using commercially available process simulation software. The paraffinic hydrocarbon-containing feed was pure ethane. In a first case the deethyleneizer column 49 was operated according to the process of this invention, in this case with a molar ratio of C3+ hydrocarbons to ethane of 0.020 in the overhead stream 53. In a second case column 49 was operated as a conventional deethanizer column wherein the molar ratio of C3+ components to ethane in the overhead stream 53 was 0.005. Table 1 provides a summary of the operation of column 49 in the two cases.

It can be seen that allowing a significant quantity of C3 hydrocarbons into the deethyleneizer overhead stream, and therefore into the C2 splitter bottoms stream, provides surprisingly large energy benefits. In particular, operating the deethyleneizer column according to this invention requires a condenser (unit 50 in FIG. 1) duty of approximately 600 kW. If this column were operated in the conventional manner, with a C3s recovery of 99% into the deethanizer bottoms, the duty of the condenser 50 would increase to over 1800 kW. The lower duty of the current invention translates to an approximately 1 MW savings in refrigerant compressor power for the current invention over the prior art. The novel combination of a deethyleneizer tower coupled with the use of an autothermal cracking reactor, as taught in the current invention, provides a means for taking advantage of this energy benefit. The optimal operating point of the deethyleneizer tower of this invention will depend on a number of factors, including the cost of energy, the composition of the deethyleneizer tower feed, and the extent to which the acetylene reactor catalyst can tolerate unsaturated C4's.

TABLE 1
Performance of Column 49 (FIG. 1)

| | Case 1 | Case 2 |
|---|---|---|
| | This | Conventional |
| | Invention | Deethanizer |
| C3+ in stream 53 (kg/hr) | 593.5 | 134.5 |
| Molar Ratio of C3+ to | 0.020 | 0.005 |
| Ethane in Stream 53 | | |
| Duty of Condenser 50 (kW) | 597 | 1820 |
| Recovery of ethylene from | 99.8% | 99.8% |
| stream 46 to stream 53 | | |
| Recovery of C3+ from | 5.99% | 1.40% |
| stream 46 to to stream 53 | | |

That which is claimed is:

1. A process for the production of ethylene from a paraffinic hydrocarbon-containing feed, said process comprising the steps of:
(i) autothermally cracking the paraffinic hydrocarbon-containing feed with a molecular oxygen-containing gas in contact with a catalyst capable of supporting combustion beyond the normal fuel rich limit of flammability to produce a product stream comprising ethylene, ethane, propylene, carbon monoxide, hydrogen, and hydrocarbons heavier than propylene,
(ii) chilling and partially condensing said product stream to produce one or more liquid streams, and an uncondensed vapor stream;
(iii) treating at least one of said one or more liquid streams in a demethanizer distillation column, to produce an overhead product comprising hydrogen, carbon monoxide and methane and a bottoms product comprising hydrocarbons heavier than methane;
(iv) treating the bottoms product comprising hydrocarbons heavier than methane from step (iii) in a deethyleneizer column, to produce an overhead product comprising ethylene, ethane, propylene and propane, with essentially no C4 hydrocarbons, and a bottoms product comprising C4 and heavier hydrocarbons,
(v) treating the overhead product comprising ethylene, ethane, propylene and propane in a C2 splitter to produce an overhead ethylene product stream and a bottoms stream comprising ethane, propylene and propane, and
(vi) recycling the bottoms stream comprising ethane, propylene and propane to the autothermal cracking step (i).

2. A process as claimed in claim 1, wherein the catalyst capable of supporting combustion beyond the fuel rich limit of flammability comprises a Group VIII metal as its catalytic component.

3. A process as claimed in claim 1, wherein the paraffinic hydrocarbon-containing feed comprises a gaseous feed selected from ethane, propane, butane and mixtures thereof.

4. A process as claimed in claim 1, wherein to produce the product stream comprising ethylene, ethane, propylene, carbon monoxide, hydrogen, and hydrocarbons heavier than propylene for subsequent separation, the cracked gas stream from the autothermal cracker is first treated to remove carbon dioxide, any residual oxygen and any residual water.

5. A process as claimed in claim 1, wherein in step (ii) the product stream comprising ethylene, ethane, propylene, carbon monoxide, hydrogen, and hydrocarbons heavier than propylene is passed to a chilling train comprising one or more stages in which chilling and partial condensation occur.

6. A process as claimed in claim 1, wherein the uncondensed vapor stream produced in step (ii), which comprises hydrogen, carbon monoxide and methane, is used as a fuel gas, is further treated to recover hydrogen and/or carbon monoxide, and/or is used as a synthesis gas feed for a subsequent chemical process.

7. A process as claimed in claim 1, wherein there are more than one liquid streams produced in step (ii) that are passed to the demethanizer column in step (iii) and they are passed to the demethanizer as separate streams, or two or more of the liquid streams are combined and passed to the demethanizer distillation column.

8. A process as claimed in claim 1, wherein the overhead product comprising hydrogen, carbon monoxide and methane from step (iii) may be combined with the uncondensed vapor stream recovered from the chilling and partial condensation in step (ii) or may separately be used, for example as a fuel gas, treated to recover hydrogen and/or carbon monoxide and/or used as a synthesis gas feed for a subsequent chemical process.

9. A process as claimed in claim 1, wherein the overhead product from the deethyleneizer column in step (iv) contains acetylene and is directed to an acetylene hydrogenation system prior to treatment in the C2 splitter of step (v).

10. A process as claimed in claim 1, wherein at least a portion of the bottoms product of the deethyleneizer column is recycled to the autothermal cracking process of step (i).

11. A process as claimed in claim 1, wherein the chilling and partial condensing of step (ii) comprise at least one rectification operation.

12. A process as claimed in claim 1, wherein at least a portion of the chilling of step (ii) is provided by a refrigeration system in which the working fluid is composed of more than one hydrocarbon component, at least two of which have a different number of carbon atoms.

13. A process as claimed in claim 1, wherein the molar ratio of hydrocarbons heavier than ethane to ethane in said deethyleneizer overhead product is greater than or equal to 0.01.

14. A process as claimed in claim 10, wherein at least 90% of the propylene in the product stream of step (i) is recycled to the autothermal cracking process of step (i).
